(19) United States
(12) Patent Application Publication
(10) Pub. No.: US 2003/0166608 A1
(43) Pub. Date: Sep. 4, 2003

(54) Polysaccharide products from durian: process for isolation and purification and their applications

(75) Inventor: Sunanta Pongsamart, Bangkok (TH)

Correspondence Address: Chulalongkorn University, Faculty of Pharmaceutical Sciences, Bangkok, 10330, TH

(73) Assignee: Chulalongkorn University, Bangkok (TH)

(21) Appl. No.: 10/085,897

(22) Filed: Mar. 1, 2002

(51) Int. Cl. A61K 31/715; C08B 37/00

(52) U.S. Cl. 514/54; 536/123

(57) ABSTRACT

A process for isolation and purification of polysaccharide gel and fiber from dried fruit-hull of Durio zibethinus L. Two polysaccharide products are obtained. The process involves hot water extraction of crude polysaccharide gel from fruit-hulls, filtration and precipitation in acid-lower aliphatic alcohol and further purification by reprecipitation of concentrated aqueous crude polysaccharide gel, after demineralization using cation-exchange resin, in acid-lower aliphatic alcohol and washing in alcohol. Dried polysaccharide gel is derived as creamy to white product. Polysaccharide gel is used as dressing-patch, film-dressing, sustained-release dressing, gelling agent; used in food preparation such as jelly, medical-food products. Fiber residue after hot water extraction is treated to isolate polysaccharide fiber. The process comprises alkali hydrolysis, acid hydrolysis, decolorization and washing in dilute acid, sodium chloride and water to derive white polysaccharide fiber. Dried product is pulverized to derive water insoluble powder. Polysaccharide fiber is used as diluent, binder and food fiber.

### INTRODUCTION

1. Field of the Invention

This invention related to products which are polysaccharides from durian and a process for isolation and purification of two polysaccharide products, polysaccharide gel and polysaccharide fiber. The elemental compositions are C, H and O in molar ratio closed to 3:6:3. Polysaccharide gel has an average molecular weight of 500-1,400 kDa, isolated products are obtained in forms of dried light fiber sheet and powder according to drying procedure. Polysaccharide gel is swelled and forms a viscous layer in water. Polysaccharide fiber is water insoluble. Polysaccharide gel and polysaccharide fiber can be applied widely in pharmaceutical and medical-food industries.

2. Background of the Invention

Polysaccharides associated with cellulose structure of plants, such as gum and pectin, are compound useful for food and pharmaceutical industries. The invention of isolating process for such value-added products from plant waste is an interesting subject for agricultural countries. Thailand is one of these countries, varieties of fruits have been cultivated and durian (Durio zibethinus L.) is the most popular fruit, durian has been accepted as “king of fruit”. Fruit of durian is now all season available agricultural product, and the consequence of fruit-hulls waste treatment being burden every year especially during its ordinary season. However, hulls of durian fruit are agricultural waste that can make it possible to derive benefit from durian waste material, and utilize agricultural product most efficiently. Value added polysaccharide products isolated from plants are useful as pharmaceutical aids. The present invention of Pongsamart involves the processes for isolation and purification of polysaccharide gel and fiber from hulls of durian fruit is to provide valuable polysaccharide products useful for pharmaceutical and food industries. The polysaccharide gel and polysaccharide fiber isolated from fruit-hull of durian was developed to receive the highest yield and satisfactory purified products. Supplying of plant material is sufficient each year, in addition the invention make value-added product from agricultural waste as well as minimize waste material for scavenger.

### SUMMARY OF THE INVENTION

It is an object of the present invention to provide two polysaccharide products and a successful methods for isolating commercially valuable polysaccharide gel and polysaccharide fiber useful for pharmaceutical and food industries. The elemental compositions present only C,H and O in atomic ratioes of 2.9:5.7:3.2 and 3.5:6.4:3.1 in polysaccharide gel and polysaccharide fiber, respectively. Sugar compositions are arabinose, rhamnose, fructose, glucose and galacturonic acid in polysaccharide gel. Polysaccharide fiber composed of glucose. Hot water extract under mild acid condition of durian fruit-hulls, which is previously washed in acid-alcohol, is clarified and filtered, concentrated and then precipitated as crude polysaccharide gel in cold acid-alcohol. Aqueous solution of the crude polysaccharide gel is again clarified and filtered, and then applied on a column of ion-exchange resin, polysaccharide gel is eluted, concentrated and precipitated a purified polysaccharide gel in cold acid-alcohol, alcohol washed and dried, powder of polysaccharide gel is soluble and forms a viscous layer in water. Products of polysaccharide gel is used for wound healing as film dressings, dressing patches, sustained-release film patches; or used as gelling agent and thickening agent in pharmaceutical preparation; and used in food preparations such as jelly and jam, diet and medical-food. Fiber residue after hot water extraction is treated to derive a polysaccharide fiber by hydrolyzing in alkali and decolorizing, hydrolyzing in acid and decolorizing, and then washing in water and in solutions of dilute acid, salt and finally with water. A white polysaccharide fiber is dried, and milled to a fine powder which is water insoluble. Product of polysaccharide fiber is used as diluent, binder and food fiber in pharmaceutical and food preparations.

### DESCRIPTION

The object of this invention has been achieved by producing two products of polysaccharide and providing a process for isolation and purification of the two forms of polysaccharides, gel and fiber forms. Fresh durian-hull is blended, dried, and powdered. Dried hull is washed in acid-lower aliphatic alcohol and followed by extraction with hot water, organic acid is added to adjust pH to 4.5-4.7. Water extract is clarified and filtered through a filter aid such as kieselguhr and diatomaceous earth. The filtrate is concentrated and viscous liquid is precipitated to obtain a polysaccharide gel in 4 volumes of cold acid-lower aliphatic alcohol, the precipitate is washed in 75% lower aliphatic alcohol and finally in 95% alcohol. The product of polysaccharide gel crude extract is dried in hot air oven or freeze dryer, and ground to powder. The crude extract of polysaccharide gel is treated to purify by reprecipitating of clear concentrated aqueous solution of polysaccharide gel, after having been demineralized using ion-exchange resin, in cold acid-lower aliphatic alcohol and washing the precipitate twice in cold 75% lower aliphatic alcohol and final washing in 95% alcohol or acetone. The precipitate of polysaccharide gel is filtered and dried, creamy to white powder or light fiber sheet product of 7-9% yield of purified polysaccharide gel is obtained which is depending on drying procedure. The fiber residue after completed hot water extraction is washed in alcohol and dried in hot air oven and being used in treatment for isolation of polysaccharide fiber.

The dried fiber residue is suspended in a solution of dilute alkali and hydrolyzed in autoclave and filtered after it had cooled to room temperature, and the fiber residue is washed with water until filtrate is neutral and dried. The dried fiber residue is decolorized in bleaching agent. The dried fiber residue is then treated to hydrolyze unwanted impurities in dilute mineral acid and autoclaved, after the mixture had cooled to room temperature the precipitate is filtered and washed with water until filtrate is free from acid and dried. Dried fiber residue is decolorized and filtered and then washed with washing solution and then with water until filtrate is free of chloride. White residue of polysaccharide fiber is final washed in 95% alcohol or acetone, and dried in hot air oven or freeze dryer. The dried polysaccharide fiber is ground to powder, 30% yield of white polysaccharide fiber is obtained from dried fiber residue.

Polysaccharide gel is utilized in pharmaceutical preparations for moist and dry wound healing such as dressing patches, film dressings, sustained-release film patches; in gel and cream preparations as gelling agent, thickening agent; and in food preparations such as jelly and jam, diet and medical-food products. Polysaccharide fiber is utilized in pharmaceutical and food preparations as diluent, binder and food fiber.

The following examples illustrate the process of isolation and purification of polysaccharide gel and polysaccharide fiber from hulls of durian fruit and their applications. However, such examples should not be interpreted as a limited on the invention.

### EXAMPLE 1

One Kg of blended or hammer milled dried fruit-hulls of durian was soaked in 15 l of 5% hydrochloric acid in 75% alcohol for 6 hrs, washed in 75% alcohol and filtered. The residue was dried at 70° C. in hot air oven. Dried residue was suspended in 24 l of hot water and 120 g sodium hexametaphosphate, added citric acid crystal to bring pH to 4.5-4.7. The mixture was heated to mild boiling for 40 minutes and filtered through sheet cloth, first filtrate was collected. Second hot water extraction was repeated by the same procedure with 15 l water, the second hot filtrate was pooled together with the first filtrate, kieselguhr (20 g/l) was added and mixed by stirring for 15 minutes to clarify the filtrate, vacuum filtered a hot mixture through a Buchner funnel, the polysaccharide gel filtrate was concentrated by rotary evaporator at 70° C. to reduce 18-20 times of total volume. A viscous liquid was mixed in 4 volumes of cold acid-ethanol (5% HCl in 75% ethanol) with continuous stirring for 20-30 minutes until precipitation was completed, the precipitate of crude polysaccharide gel was collected by filtering through fine nylon sheet, the precipitate was washed once in the same solvent and then washed twice in cold 75% ethanol and filtered and pressed to remove excess solvent, and dried by final washing with 95% ethanol or acetone, filtered and pressed to remove an excess solvent. Crude extract of polysaccharide was dried in hot air oven at 70° C. or in freeze dryer. The dried crude extract was ground to powder. A light-brown powder of 20% yield was obtained. Fiber residue after second hot water extraction was dried by washing with alcohol and then drying in hot air oven at 70° C. Dried fiber residue was ground and used as a material for isolation of polysaccharide fiber.

### EXAMPLE 2

The polysaccharide gel crude extract in Example 1 was dissolved in hot water to 1% solution, filtered by vacuum filtration through purified kieselguhr (10 g/l) in Buchner funnel, then a clear filtrate was applied on a column of cation-exchange resin and eluted polysaccharide gel was concentrated by rotary evaporator at 70° C. to reduce total volume. A clear viscous liquid of polysaccharide gel was treated in 4 volumes of cold acid-ethanol (5% HCl in 75% ethanol). White precipitate was collected by filtration through fine nylon sheet, washed the precipitate twice in cold 75% ethanol or until the filtrate was acid freed, filtered and removed excess solvent, and then dried by final washing in 95% ethanol, filtered and pressed to remove excess solvent. A purified polysaccharide gel was dried in hot air oven at 70° C. or in freeze dryer. The polysaccharide gel was ground to powder of white or creamy color, 7-9% yield of purified polysaccharide gel was obtained. Dried powder of polysaccharide gel was swelled and formed a viscous layer in water, the viscous liquid had acid pH at 2.5±2. An aqueous solution of 3% polysaccharide gel has viscosity at 200-350 centripoies.

### EXAMPLE 3

Example 3 was a purified polysaccharide gel as Example 2 except that the polysaccharide gel after final wash was dissolved in water to 0.5-1% solution and followed by drying in freeze-dryer. A product of white, light and thick fiber sheet of purified polysaccharide gel was obtained. The product was easily dissolved in water. A ground product was a very light powder and easily dissolved in water.

### EXAMPLE 4

Example 4 was a purified polysaccharide gel as Example 2 except that the polysaccharide gel after final wash was dissolved in water to 1-1.5% solution and dried in spray-dryer. A creamy white fine powder of polysaccharide gel product was obtained. Dry powder dissolved faster in water.

### EXAMPLE 5

The dried fiber residue of dried hulls after polysaccharide gel extraction in Example 1 was treated to isolate polysaccharide fiber by the following procedure. One Kg of dried fiber residue was suspended in 0.36 M 10 l of sodium hydroxide, hydrolyzed in autoclave at 121° C., 15 lbs/in² pressure for 30 minutes. A suspension was cool to room temperature and filtered in Buchner funnel, washed with water until the filtrate was neutral, pH closed to 7. Dried fiber residue was decolorized in 10 l of sodium hypochlorite (4% chlorine) for 1-2 days and washed with water. The dried fiber residue was then treated in 1.8 M, 10 l, hydrochloric acid and hydrolyzed unwanted impurities in autoclave at 121° C., 15 lbs/in² pressure for 30 minutes. A suspension mixture was cooled to room temperature and filtered, the fiber residue was washed with water until the filtrate was neutral. The fiber residue was dried and decolorized in 10 l of sodium hypochlorite (4% chlorine) for 1-2 days. The mixture was filtered in Buchner funnel and washed with water until no odor of chlorine, washed in dilute acid and in 0.5 M, 10 l of sodium chloride for 3-4 hrs, filtered and washed with dilute acid and then water until filtrate was free from chloride. White fiber residue was dried by final washing with 95% ethanol, pressed to remove excess ethanol, and dried in hot air oven. A dried product of polysaccharide fiber was ground, white powder of 30% yield of the polysaccharide fiber from dried fiber residue was obtained. Dried powder was water insoluble.

### EXAMPLE 6

Product of purified polysaccharide gel in a form of white, light and thick fiber sheet after freeze-drying in EXAMPLE 3 was cut into pieces of wound dressing and sterilized for using as a dressing patch for healing moist and dry wounds.

### EXAMPLE 7

Powder of polysaccharide gel in EXAMPLE 2, 3 and 4 was added with plasticizer in aqueous solution such as glycerin, propylene glycol and polyethylene glycol and forming films by casting/solvent evaporation method. Sterilized film-dressing was used for healing moist and dry wounds. Preparation of film-dressing with drugs was applied as a sustained-release film dressing.

### EXAMPLE 8

Powder of polysaccharide gel in EXAMPLE 2, 3 and 4 was used in pharmaceutical preparations as a gelling agent and thickening agent in gel and cream products, and as a binder and disintegrator in tablet. It was also used in food preparations such as jelly and jam, diet and medical-food products for diabetic, high cholesterol and obese patients.

### EXAMPLE 9

Powder of polysaccharide fiber in EXAMPLE 5 was used as a diluent, binder and food fiber in pharmaceutical and food preparations.

What I claimed is:

1. Two products of polysaccharides, polysaccharide gel and polysaccharide fiber, isolated from durian (Durio zibethinus L.), both products are prepared in forms of dried powder and dried fiber sheet.

2. A process for isolation of polysaccharides from parts of durian (Durio zibethinus L.) to derive two polysaccharide forms, a polysaccharide gel and a polysaccharide fiber as in claim 1, wherein isolation of polysaccharide gel comprises blending fresh hull or blending or pulverizing dry hull of durian fruit, hot water extracting process of polysaccharide gel in the presence of organic acid to bring pH to 4.5-4.7, clarifying hot filtrate of water extract by mixing with clarifying agent, precipitating of concentrated viscous polysaccharide water extract in 4 volume of cold acid-lower aliphatic alcohol, washing in cold 75% alcohol and final washing in 95% alcohol to dehydrate precipitate of crude polysaccharide gel and drying in hot air oven at 70° C., crude polysaccharide gel is further purified by repeating precipitation of clear viscous concentrated solution of polysaccharide gel eluted from a column of cation-exchange resin in 4 volumes of cold acid-lower aliphatic alcohol, washing resulting precipitate twice in cold 75% alcohol and finally in 95% alcohol to dehydrate a purified polysaccharide gel, and drying in hot air oven or lyophilizer.

3. A process for isolation of polysaccharides from parts of durian (Durio zibethinus L.) to derive two polysaccharide forms, a polysaccharide gel and a polysaccharide fiber, as in claim 1, wherein isolation of polysaccharide fiber comprises treating fiber residue after polysaccharide gel extraction in 0.36 M sodium hydroxide, hydrolyzing in autoclave for 30 minutes, washing with water and decolorizing with decolorizing agent, treating resulting white residue in 1.8 M mineral acid to hydrolyze unwanted impurities in autoclave for 30 minutes, washing residue of polysaccharide fiber with water, decolorizing and washing white polysaccharide fiber with water, and with washing solutions comprising dilute acid, sodium chloride and water, successively, until filtrate is free of chloride, white polysaccharide fiber is dried by suspending in 95% alcohol, filtered and dried in hot air oven or freeze dryer.

4. The process as in claim 2 wherein water extract is clarified using clarifying agent and filter aid such as kieselguhr or diatomaceous earth.

5. The process as in claim 2 wherein drying process to obtain purified polysaccharide gel are spray drying or freeze-drying of 0.5-1.5% aqueous solution of polysaccharide gel.

6. The process as in claim 3 wherein said washing solutions for said washing process after acid hydrolyzing and decolorizing procedure, are 0.1 M hydrochloric acid and 0.5M sodium chloride, successively.

7. The applications of polysaccharide gel according to claim 1 for moist and dry wound healing as dressing-patch, film-dressing, sustained-release film dressing, utilization in pharmaceutical preparations as gelling agent, thickening agent, utilization in food preparation such as jelly and jam, diet and medical-food products.

8. The applications of polysaccharide fiber according to claim 1 as diluent, binder, food fiber in pharmaceutical and food preparations.
